(19) United States
(12) Patent Application Publication
(10) Pub. No.: US 2025/0281214 A1
(43) Pub. Date: Sep. 11, 2025

(54) BIO-RESORBABLE ORTHOPAEDIC LAG SCREW FOR SMALL BONE FRACTURE FIXATION

(71) Applicant: Biocomposites Limited, Keele (GB)

(72) Inventors: Harley Jay Williams, Weston (GB); John Warren Colclough, Stoke-on-Trent (GB); Phillip Anthony Laycock, Winterley (GB)

(73) Assignee: Biocomposites Limited, Keele (GB)

(21) Appl. No.: 18/606,693

(22) Filed: Mar. 15, 2024

(30) Foreign Application Priority Data
Mar. 5, 2024 (GB) 2403196.5

(51) Int. Cl. A61B 17/86; A61B 17/68; A61F 2/30; A61L 27/46

(52) U.S. Cl. CPC A61B 17/8685; A61F 2/30; A61L 27/46; A61B 2017/681; A61F 2002/30062

(57) ABSTRACT

The present invention relates to a bio-resorbable calcium composite lag screw to achieve compression in small bone fractures. The design features allow for reliable insertion reducing the risk of fracture by having an internal geometry of the screw wherein an inserter or driver used to insert the screw applies force only to the threaded portion of the screw distal from the inserter or driver, reducing the risk of screw breakage on insertion.

### FIELD OF THE INVENTION

The present invention relates to a bio-resorbable screw to achieve compression in small bone fractures to aid in the repair thereof. The design features of the invention allow for reliable insertion of the screw, reducing the risk of fracture of the screw by having an internal geometry of the screw wherein an inserter or driver used to insert the screw applies force only to the threaded portion of the screw distal from the inserter or driver, reducing the risk of screw breakage on insertion. The screw may be, for example, a calcium composite lag screw.

### BACKGROUND OF THE INVENTION

Internal fixation of small bone fractures is conventionally achieved with the use of traditional hardware such as pins, plates, wires and screws including compression screws. Many of these are limited to open surgery. For simple fractures, percutaneous procedures are possible by using compression screws. These are either fully threaded variable pitch compression (VPC) screws or lag screws with a partial thread at the distal end of the screw with a conical head at the proximal end to achieve compression.

The use of polymer or metal screws can produce a foreign body response involving fibrous encapsulation of the implant, which can inhibit bone formation at the implant site. For polymer screws, this fibrous layer encapsulating the implant has the potential to weaken the fracture site and increase the risk of further fracture or non-union. Polymer screws are typically manufactured from polylactic acid, and dissolution of the screw in the body can create an acidic environment detrimental to the fracture site. Associated complications with metal screws include stress shielding within the bone, leading to a loosening of the implant and a reduction of local bone density, which can be problematic in fracture fixation. In addition, the presence of metal screws can present imaging artifacts when using magnetic resonance imaging (MRI) or computed tomography (CT).

Bioabsorbable screws manufactured from ceramic composites overcome some of the issues with polymer and metal screws. The use of a ceramic composite can provide a modulus closely matched to bone, reducing stress shielding, does not affect imaging by MRI or CT, and the combination of a calcium ceramic with a polymer may also act as a pH buffer, thus reducing the risk of acidosis. Finally, calcium composite based screws are safely absorbed by the body and are replaced by bone, preventing technical challenges for future revisions. In contrast, metal screws may require surgical removal.

However, one significant issue with ceramic composite screws is that they are mechanically weaker than the equivalent metal screws and can break when subjected to high rotational forces during insertion.

Percutaneous insertion of current compression screws utilises a guidewire to allow for accurate placement, and the screws are therefore fully cannulated. The internal geometry of the screw must allow the screw to be fully driven into the fracture site until the conical head of the screw is flush with the cortical bone to achieve compression. High insertion torque has the potential to twist the insertion tool once the threaded position of the screw is tight. However, this rotational force on the screw shaft can result in breakage of the screw.

There is therefore a need to provide a bio-absorbable compression screw which is able to resist breakage when exposed to a rotational force when inserting the screw. The present invention achieves this aim.

### DETAILED DESCRIPTION OF THE INVENTION

Therefore, provided in conjunction with the present invention is a bio-absorbable screw able to compress two fracture fragments together, the screw comprising a body having a threaded outer section, and an axial longitudinal opening extending therethrough, the opening comprising (i) a square internal geometry at an internal position within the screw corresponding to the threaded outer section, the square internal geometry extending between 20% to 100% of the length of the threaded outer section of the screw; and (ii) a circular internal geometry for the remaining length of the screw.

It has been found that the design of the screw's internal geometry and associated instruments such as an inserter/driver can influence the forces acting on the screw during insertion and can be adapted to reduce the risk of fracture. The square internal geometry of the screw drives the threaded portion of the screw where the insertion torque is at its greatest. The remainder of the longitudinal opening is circular/cylindrical in geometry to allow a universal inserter/driver for different screw lengths and prevent rotational forces acting on the non-threaded shaft portion of the screw.

The inserter/driver is inserted fully into the screw, such that the end of the inserter/driver engages with the section of the screw having a square internal geometry. The force from the inserter/driver is thus applied only to the threaded distal section of the screw through the presence of the square geometry internal to the screw that is present over 20% to 100% of the length of the threaded outer section of the screw. It is this which greatly reduces the risk of the screw breaking when exposed to the rotational forces.

By way of comparison, although they are fully cannulated, metal compression screws are inserted with the inserter/driver located in the proximal end only. Force is thus applied to this end of the screw. Polymer compression screws also have a full cannulation, but the inserter/driver only locates halfway inside the screw.

### DESCRIPTION OF PREFERRED EMBODIMENTS

The screw is typically a lag screw. The length of the screw may be any length suitable to compress two fracture fragments together, which will of course differ depending upon which bone has been fractured. For example, it may be between about 14 mm and about 50 mm, depending upon the fracture to be repaired, and a suitable length will be apparent to a skilled medical practitioner.

The screw typically has a conical head distal to the threaded portion. The conical head has an opening therein which is circular in shape. This is to allow an elongated distally cylindrical part of the inserted inserter/driver to extend about 0.2 mm out from the tip of the screw, to protect the end of the screw due to the more brittle nature of the bio-absorbable material the screw is made from.

The length of the screw is comprised of a shaft portion which has a circular internal cross-section, and a threaded portion which has a square internal cross-section. The square internal geometry extends between 20% to 100% of the threaded outer section of the screw, typically 30 to 90%, more typically 40-80%, more typically 50-70% of the threaded outer section of the screw.

The outer diameter of the shaft portion—i.e. the distance from a first point on the external surface of the shaft portion to a second point on the external surface of the shaft portion diametrically opposed to the first point is typically between about 3 mm and about 7 mm.

The inner diameter of the shaft portion—i.e. the distance from a first point on the internal surface of the shaft portion to a second point on the internal surface of the shaft portion diametrically opposed to the first point is typically between about 2 mm and about 6 mm.

The screw is made of a material that is bioabsorbable. It may comprise or substantially consist of a material such as a bioabsorbable polymer. Non-limiting examples of bioabsorbable polymers which could be used with the present invention include poly(L-lactide) acid (PLLA), poly(D-lactide) acid (PDLA), polylactic acid (PLA), or a combination of any two or more thereof. The screw may also comprise an amount of a non-bioabsorbable polymer, such as a metal or polyether ether ketone, The screw may also comprise a bioactive filler in addition to the bioabsorbable material, such as the bioabsorbable polymer. Non-limiting examples of a bioactive filler which could be used with the present invention include, for example, hydroxyapatite, beta-tricalcium phosphate, or calcium sulfate, or a combination of any two or more thereof.

The inserter/driver typically comprises a square geometry at one end designed to engage with the square internal geometry of the threaded section of the screw. Either side of this square geometry, the inserter/driver typically tapers to a cross-section that will fit within the void but not apply force, such as a circular cross-section.

Also provided in accordance with the present invention is a method of inserting a bioabsorbable screw to compress two fracture fragments together using an inserter/driver;
the screw comprising a body having a threaded outer section, and an axial longitudinal opening extending therethrough, the opening comprising (i) a square internal geometry at an internal position within the screw corresponding to the threaded outer section, the square internal geometry extending between 20% to 100% of the length of the threaded outer section of the screw, and a circular internally geometry for the remaining length of the screw; and
the inserter/driver comprising a square geometry at one end configured to engage only with the square internal geometry of the threaded section of the screw, the inserter/driver tapering to a reduced cross-section (such as a circular cross-section) both distally and proximally of the square geometry.

Also provided in accordance with the present invention is a kit of parts for the insertion of a bioabsorbable screw to compress two fracture fragments together, the kit comprising:
i) a bio-absorbable screw which is able to compress two fracture fragments together, the screw comprising a body having a threaded outer section, and an axial longitudinal opening extending therethrough, the opening comprising (i) a square internal geometry at an internal position within the screw corresponding to the threaded outer section, the square internal geometry extending between 20% to 100% of the length of the threaded outer section of the screw, and a circular internally geometry for the remaining length of the screw; and
ii) an inserter/driver comprising a square geometry configured to engage only with the square internal geometry of the threaded section of the screw, the inserter/driver tapering to a reduced cross-section (such as a circular cross-section) both distally and proximally of the square geometry.

The kit of parts may also comprise one or more further components selected from a guide wire, a tap, a drill and a soft tissue protector/guide wire inserter. These are all further devices which can aid in the positioning and fixing of a screw for the purpose of the invention.

When positioning the screw for insertion, a medical practitioner will typically reduce the fracture/osteotomy and select an optimum insertion point. A non-limiting example of a mode of inserting the screw of the invention is described below.

A guide wire, sharp end first, is typically inserted through a central hole in a guide wire sleeve, and is advanced to a minimum of 10 mm beyond the fracture/cut line. The position is confirmed by X-ray.

The guide wire sleeve should then be removed, and a depth gauge inserted over the central guide wire to the cortical bone surface, and its position confirmed on X-ray, and. the depth of the guide wire from a laser mark (not from the end) on the guide wire should be measured and recorded. Once the desired depth is recorded, advance the guide wire to prevent pull out at later stages of the procedure.

The drill bit should be advanced over the guide wire to the pre-recorded depth, and the tap inserted to the pre-recorded depth, but no further. Then select a screw that is 2 mm shorter than the pre-recorded depth and mount it on the inserter/driver. Advance slowly over the central guide wire. When compression is achieved, the inserter/driver and guide wires should be removed, and the wound closed.

If the screw head is sitting proud of the cortical bone, effective compression will not be achieved. To avoid this, ensure the correct depth measurement is taken. The depth gauge should be in contact with the cortical bone surface (checked on X-ray) and the guide wire at the desired depth of the screw. If the depth gauge has been measured incorrectly or the bone has not been drilled and tapped to the required depth, resistance may be encountered when turning the screw due to the screw threads contacting untapped bone. If this happens, one should use a shorter screw, or drill and tap the bone to the correct depth.

### BRIEF DESCRIPTION OF THE FIGURES

FIG. 1 illustrates the distal end of the screw inserter/driver featuring a square geometry in line with the threaded section of the screw tapering to a circle both distally and proximally and a proximal section locating within the screw mid shaft having a circular cross section.

FIG. 2 illustrates a cross section demonstrating the screw inserter/driver fully locating in the bioabsorbable lag screw.

FIG. 3 illustrates the bioabsorbable lag screw located on the screw inserter/driver.

FIG. 4 illustrates the bioabsorbable lag screw located on the screw inserter/driver with the screw transparent for visualization.

FIG. 5 illustrates the bioabsorbable lag screw in cross section demonstrating the internal geometry within the threaded section of the screw.

FIG. 6 illustrates the bioabsorbable lag screw demonstrating the circular proximal opening of the screw.

FIG. 7 illustrates a cross section of the bioabsorbable lag screw located in the screw inserter/driver.

FIG. 8 illustrates a cross section of the bioabsorbable lag screw.

### DETAILED DESCRIPTION OF THE FIGURES

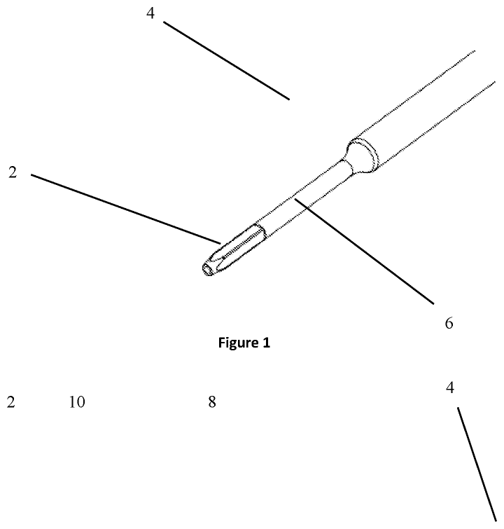

FIG. 1 shows the distal end 2 of the screw inserter/driver 4 featuring a square geometry in line with the threaded section of the screw (not shown this Figure) tapering to a circular cross-section both distally and proximally, and a proximal section 6 to be located within the screw shaft portion having a circular cross-section.

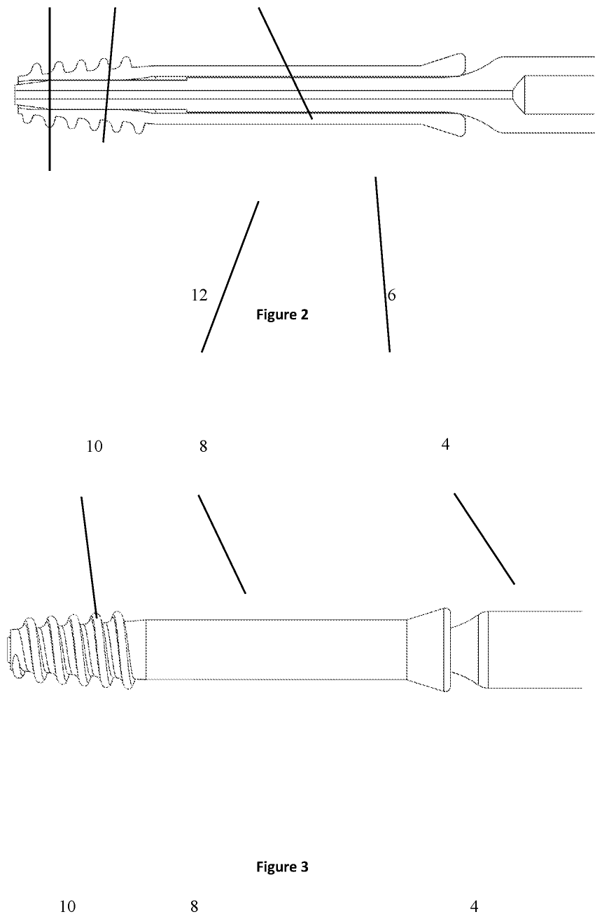

FIG. 2 shows a cross-sectional side-on view demonstrating the screw inserter/driver 4 fully located in the bioabsorbable lag screw 8. The position of the distal end 2 with the square geometry of the screw inserter/driver 4 corresponds with the square internal geometry of the screw 8 and also the threaded outer section 10 of the screw 8, and the proximal section 6 lies within the shaft portion 12 of the screw 8.

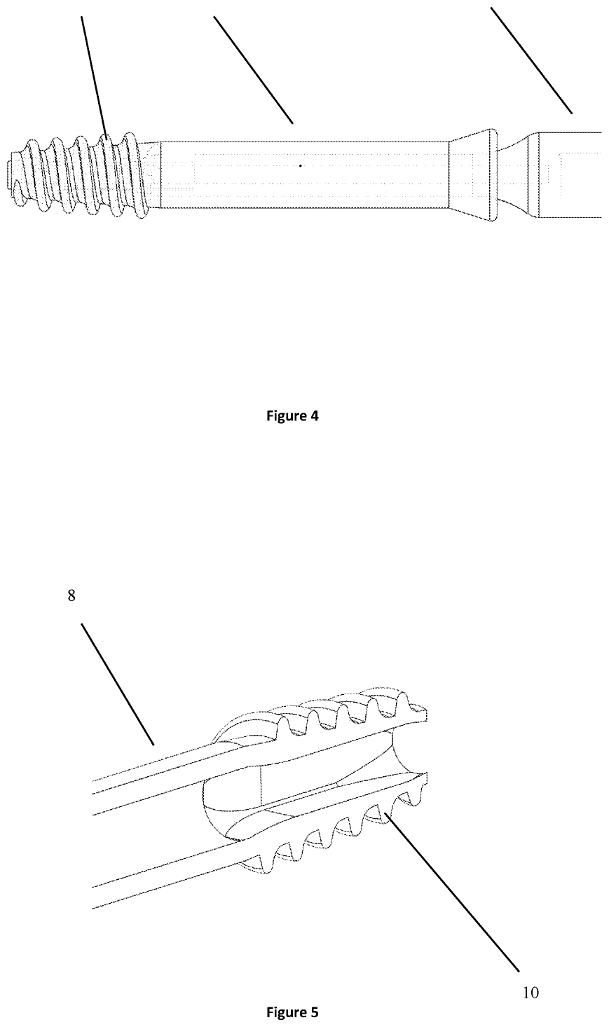

FIG. 3 shows a normal side-on view of the screw inserter/driver 4 fully located in bioabsorbable lag screw 8; while FIG. 4 also shows the screw inserter/driver 4 fully located in bioabsorbable lag screw 8, but with the screw 8 being transparent for ease of visualization.

FIG. 5 shows a cross-sectional view of the bioabsorbable lag screw 8, depicting the square internal geometry within the threaded section 10 of the screw 8. It can be seen that the internal geometry of the threaded section 10 is square in shape.

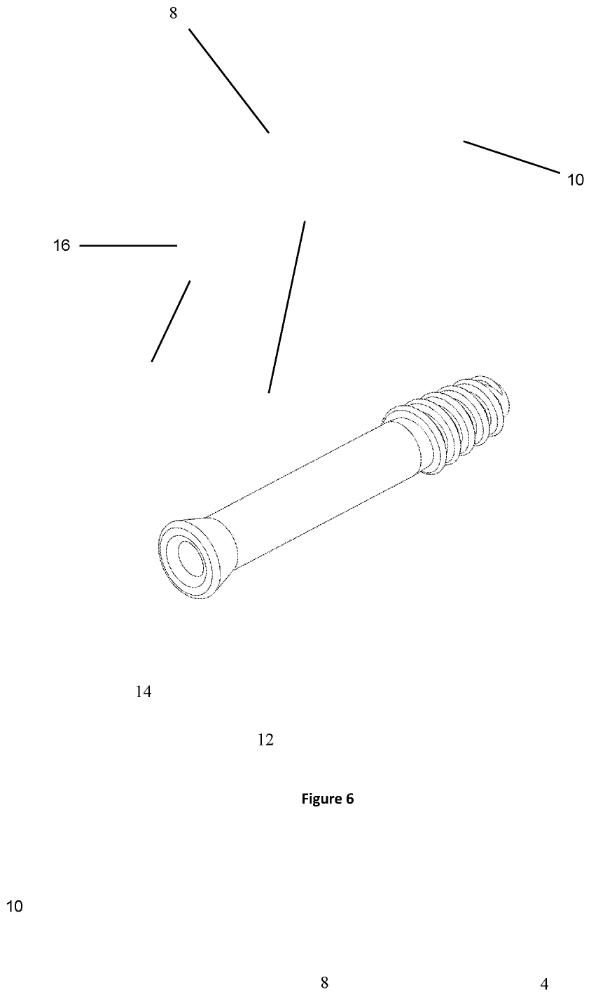

FIG. 6 shows the bioabsorbable lag screw 8 with its conical head 14 having its circular proximal opening 16 leading to the cylindrical shaft portion 12 and the threaded outer section 10.

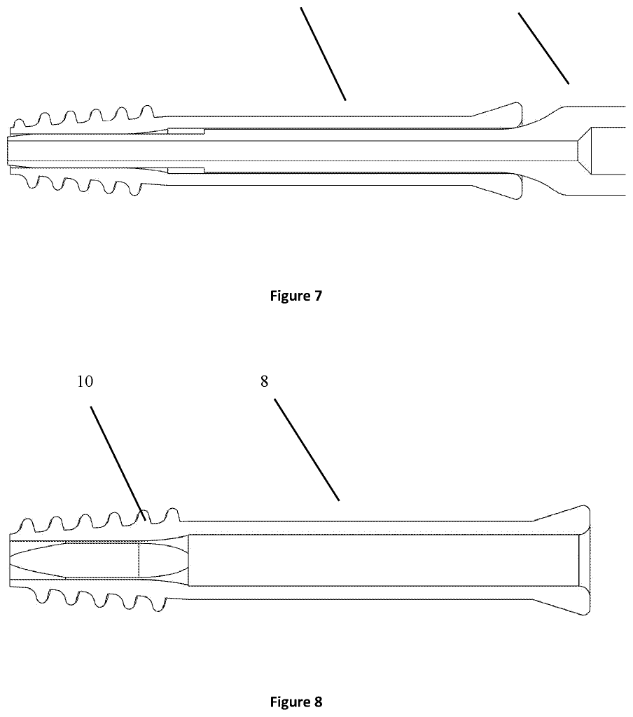

FIG. 7 shows a diagrammatical cross-section of the screw inserter/driver 4 located in the bioabsorbable lag screw 8.

FIG. 8 shows a diagrammatical cross-section of the bioabsorbable lag screw 8 without the screw inserter/driver 4 located therein.

### EXAMPLES

A rigid polyurethane (PU) foam with a density of 25 pounds per cubic foot (PCF) was used as a bone analogue material. A hole was drilled to 2 mm longer than the depth of the compression screw using guidewire and a depth gauge. The hole was tapped using a depth gauge to ensure the correct tap location. The tap was removed turning anticlockwise and the hole was cleared of loose material. The proximal part of the hole was countersunk to fit the head of the screw. Different screw samples were then inserted into the hole and overtightened until failure was observed. The results are provided in Table 1 below:

TABLE 1

| | 25 PCF Sawbone | | |
|---|---|---|---|
| | | Maximum | |
| | | Torque | |
| | Screw | (Nm) | Failure Mode? |
| A | 26 × 3.5 Full length single flat | 0.33 | Screw Break |
| | (comparison) | | Midshaft |
| | [symbol] | | |
| B | 26 × 3 full length rectangular | 0.456 | Screw Break |
| | (comparison) | | Midshaft |
| | [symbol] | | |
| C | 26 × 3.5 Square distal thread | 0.71 | No screw break |
| | (invention) | | |
| | [symbol] | | |

Screw A is a comparative screw which has a mostly circular internal geometry for the entire length of the screw, with the exception of part of the circle having a flat surface, as shown in the diagram in Table 1. This screw failed with a torque of only 0.33 Nm, with the screw breaking at its midshaft.

Screw B is another comparative screw which has a rectangular internal geometry for the entire length of the screw, as shown in the diagram in Table 1. This screw also failed, with a torque of only 0.456 Nm, with the screw again breaking at its midshaft.

In contrast, Screw C, which is a screw according to the present invention, has (i) a square internal geometry at an internal position within the screw corresponding to the threaded outer section, the square internal geometry extending between 20% to 100% of the length of the threaded outer section of the screw; and (ii) a circular internal geometry for the remaining length of the screw, as discussed herein above.

This screw did not break, even when being exposed to a torque of 0.71 Nm, significantly higher than the torques at which the comparison Screws A and B had failed and broken.

Therefore, the screw according to the present invention clearly demonstrates that it can withstand a significantly higher level of torque without suffering any midshaft failure.

The bioabsorbable screw of the present invention is able to resist breakage when exposed to a rotational force when it is being screwed into position to compress and help repair a fracture. This is clearly of benefit to patients with broken bones requiring repair. It also avoids the disadvantages detailed above for metal and non-bioabsorbable screws.

It is of course to be understood that the present invention is not intended to be restricted to the foregoing examples which are described by way of example only.

1. A bioabsorbable screw able to compress two fracture fragments together, the screw comprising a body having a threaded outer section, and an axial longitudinal opening extending therethrough, the opening comprising (i) a square internal geometry at an internal position within the screw corresponding to the threaded outer section, the square internal geometry extending between 20% to 100% of the length of the threaded outer section of the screw; and (ii) a circular internal geometry for the remaining length of the screw.

2. A bioabsorbable screw according to claim 1, wherein when the screw is subjected to a rotational force, the force is applied only to the threaded section of the screw via the square internal geometry of the screw.

3. A bioabsorbable screw according to claim 1, wherein the square internal geometry extending between 40% to 80% of the length of the threaded outer section of the screw.

4. A bioabsorbable screw according to claim 1, wherein the screw has a circular opening distal from the threaded outer section, to allow an elongated cylindrical part of the inserter/driver to extend about 0.2 mm from the circular opening of the screw.

5. A bioabsorbable screw according to claim 1, wherein the length of the screw is between 14 mm and 50 mm.

6. A bioabsorbable screw according to claim 1, wherein the outer diameter of the shaft portion is between 3 mm and 7 mm.

7. A bioabsorbable screw according to claim 1, wherein the inner diameter of the shaft portion is between 2 mm and 6 mm.

8. A bioabsorbable screw according to claim 1, wherein the screw comprises a bioabsorbable polymer.

9. A bioabsorbable screw according to claim 1, wherein the screw consists substantially of a bioabsorbable polymer.

10. A bioabsorbable screw according to claim 8, wherein the bioabsorbable polymer is selected from poly(L-lactide) acid (PLLA), poly(D-lactide) acid (PDLA), polylactic acid (PLA), or a non-bioabsorbable polymer such as polyether ether ketone, or a combination of any two or more thereof.

11. A bioabsorbable screw according to claim 8, wherein the screw comprises a bioabsorbable polymer and a bioactive filler.

12. A bioabsorbable screw according to claim 11, wherein the bioactive filler is selected from hydroxyapatite, beta-tricalcium phosphate, or calcium sulfate, or a combination of any two or more thereof.

13. A bioabsorbable screw according to claim 1, wherein the screw is a lag screw.

14. A method of inserting a bioabsorbable screw to compress two fracture fragments together using an inserter/driver;
the screw comprising a body having a threaded outer section, and an axial longitudinal opening extending therethrough, the opening comprising (i) a square internal geometry at an internal position within the screw corresponding to the threaded outer section, the square internal geometry extending between 20% to 100% of the length of the threaded outer section of the screw, and a reduced internal geometry for the remaining length of the screw; and
the inserter/driver comprising a square geometry at one end configured to engage only with the square internal geometry of the threaded section of the screw, the inserter/driver tapering to a reduced cross-section both distally and proximally of the square geometry.

15. A method according to claim 14, wherein when the screw is subjected to a rotational force, the force is applied only to the threaded section of the screw via the square internal geometry of the screw.

16. A kit of parts for the insertion of a bioabsorbable screw to compress two fracture fragments together, the kit comprising:
i) a bio-absorbable screw which is able to compress two fracture fragments together, the screw comprising a body having a threaded outer section, and an axial longitudinal opening extending therethrough, the opening comprising (i) a square internal geometry at an internal position within the screw corresponding to the threaded outer section, the square internal geometry extending between 20% to 100% of the length of the threaded outer section of the screw, and a circular internally geometry for the remaining length of the screw; and
ii) an inserter/driver comprising a square geometry configured to engage only with the square internal geometry of the threaded section of the screw, the inserter/driver tapering to a circular cross-section both distally and proximally of the square geometry.

17. A kit of parts according to claim 16, further comprising one or more selected from a guide wire, a tap, a drill and a soft tissue protector/guide wire inserter.
